(19) United States
(12) Patent Application Publication
(10) Pub. No.: US 2012/0215210 A1
(43) Pub. Date: Aug. 23, 2012

(54) APPARATUS AND METHODS FOR THE TREATMENT OF HUMAN OR ANIMAL TISSUE BY LIGHT

(75) Inventors: Stephen Brown, Gloucestershire (GB); Kevin Herbert, Gloucestershire (GB)

(73) Assignee: THE DEZAC GROUP LIMITED, Gloucestershire (GB)

(21) Appl. No.: 13/505,087

(22) PCT Filed: Nov. 1, 2010
(86) PCT No.: PCT/GB2010/051827

§ 371 Date: Apr. 30, 2012

(30) Foreign Application Priority Data
Oct. 30, 2009 (GB) 0919031.5

(51) Int. Cl. A61B 18/18

(52) U.S. Cl. 606/9

(57) ABSTRACT

Apparatus for the treatment of human or animal tissue by light includes a light source for directing light towards the tissue in use, an applicator having a tissue-facing surface, a detector for detecting in use proximity of the tissue-facing surface to the tissue, and a control element responsive to the detector for controlling the light source to direct light to be incident on the tissue in use only when the tissue-facing surface is spaced from the tissue.

This invention relates to the treatment of human or animal tissue by light and, in particular, but not exclusively, for the removal of hair.

The term ‘light’ in this specification is used to mean coherent and non-coherent light in the electromagnetic spectrum, and in both continuous wave and pulsed forms.

It is known to employ light treatment for the removal of hair from skin. The term hair removal is used to cover methods in which the hair is removed instantaneously and also, more commonly, those methods in which the life cycle of the hair follicle is interrupted so that the hair then falls out later. In a known hair removal process light or laser radiation within a specific band or at a particular wavelength selected such that the radiation is absorbed preferentially by melanin in the follicle causes local heating of the hair to a temperature which causes it to stop growth. Although the laser radiation is also absorbed in part by the skin, the heating effect is less. However, it is also important to allow dissipation of heat from the skin to ensure that there are no adverse heating effects. Therefore in a number of such treatments an applicator head is applied to the skin and light is directed onto the skin by means of a solid optical wave guide in contact with the skin. The wave guide provides a cooling effect which may be simply passive, with the wave guide being made of a material having suitable thermal conductivity properties to conduct heat away from the skin, and/or the wave guide may be actively cooled. Either way, the wave guide is kept in contact with the skin during light treatment.

There are a number of problems with this approach. In order to provide a wave guide having the required optical and thermal conductivity properties a specialist material such as sapphire has to be used at great expense. The contact with the wave guide means that the covered treatment site does not dissipate heat well and indeed certain heat transfer mechanisms such as convection are inhibited. Also, because the treatment mechanism in the prior art device is used to deposit heat energy into the hair and hair follicle, the area needs to be shaved prior to use otherwise lengths of hair projecting from the skin may be flattened against the skin as the wave guide passes over and act as heat absorbers whilst flattened against the skin thereby burning the skin. With the wave guide in contact with the skin, debris can form on the wave guide after burning hairs, potentially causing charring. The charring can generate more heat at the skin surface which is undesirable and dissipates the energy intended for treatment. This can lead to a damaged or dangerous product.

Furthermore, when the device is in full contact with the skin it makes it difficult to treat awkward areas such as the underarm armpit area, or single or small targets. Likewise, it is difficult to treat adjacent areas consistently when the wave guide has to be placed to activate the treatment light, and then removed. Furthermore, as the earlier devices tend to be pressed against the skin, the pressing or contact of the device against the skin inhibits blood flow through the skin capillaries thereby disrupting operation of the body's own cooling mechanism.

Our studies have indicated that many of these difficulties may be overcome or at least mitigated by providing a device in which an air gap is maintained between the treatment apparatus and the skin to allow dissipation of the heat generated in the skin.

JP2005102931A discloses a laser element for irradiating the skin with a beam of radiation or a wavelength which produces a vasodilatory effect. In this device the skin as a whole is the target for absorption of energy rather than preferential absorption by melanin in the hair follicles and the device is designed so that, where there are surface wounds such as a trauma, skin ulcer or the like which means that contact with the affected part should be avoided, the device can be separated above the affected part. The device includes distance sensors which allow the distance and angle of the head relative to the skin to be computed and used as inputs to a controller which permits operation of the laser beam when the distance and angles are within preset ranges, so as to ensure that a suitable treatment energy density is maintained and also that the device is not tilted to an extent that radiation enters the eye of the user. This device is concerned with maintaining sufficient energy density levels in a process in which heating of the skin as a whole is required and does not address the problem of ensuring that sufficient energy dissipates to actively avoid undue heating of the skin in a treatment in which light or radiation is preferentially absorbed by melanin in the hair follicle.

Accordingly, in one aspect, this invention provides apparatus for the treatment of human or animal skin by light to remove hair which includes:

an applicator having a skin-facing surface;

a light source for directing light towards the skin in use;

a detector for detecting in use proximity of said skin-facing surface to the skin, and

control means responsive to said detector for causing light from said light source to be incident on the skin only when the skin-facing surface is spaced from the skin.

The control means may be permissive or preventive, so that it may enable directing light when the generator is spaced from the skin, or it may inhibit operation when the generator is in contact with the skin.

In this way, an air gap is maintained between the skin-facing surface and the skin when the light source is operational. This means that any heat build up in the skin tissue can dissipate by convection, radiation as well as by the normal body mechanism for temperature control.

Although it would be possible for the control means only to monitor the minimum distance between the skin and the skin-facing surface, it is preferred for it to enable operation of the light source only when said skin-facing surface is within a preset range of distances from the skin. The preset range of distances is conveniently between 0.1 mm and 5 mm, preferably between 1 mm and 4 mm, and ideally between 2 mm and 3 mm. Thus the upper limit of the range is preferably between 3 mm and 5 mm. The lower limit is preferably between 0.1 mm and 3 mm. Moreover, the distance may be selected to ensure good if not optimal convection. There is a distance at which heat transfer by convection is a maximum. Reducing this distance impairs the heat transfer, but increasing it has little or no effect and instead compromises safety.

The apparatus preferably includes an indicator for providing an audible or visual indication of the proximity of the skin-facing surface and the skin. This may be in the form of a tone that varies in frequency and/or changes from continuous to intermittent or vice versa to indicate when the device is in range.

The proximity detector is preferably a non-contact sensor, conveniently selected from the group comprising heat sensors, capacitative sensors, optical sensors, sound or ultrasound based sensors, microwave sensor, but a contact sensor that ensures that the adjacent surface of the apparatus does not touch the skin may also be used.

In another aspect, this invention provides apparatus for the treatment of human or animal tissue by light, which includes:

an applicator having a tissue-facing surface:

a light source, and

means for maintaining a substantially unenclosed air gap of between said issue-facing surface and said tissue.

The means for maintaining may include one or more spacer elements. It conveniently maintains an air gap in the range of between 0.1 mm and 5 mm, preferably between 1 mm and 4 mm, and ideally between 2 mm and 3 mm. The means for maintaining may include a contact sensor to inhibit operation of the device when one or more spacer elements are not in skin contact.

The apparatus in either aspect may conveniently include means for detecting and/or classifying the nature of the tissue prior to treatment, such as skin type/colour, hair type/colour etc. This may be a sensor which is also operable to detect the distance between the device on the skin.

The apparatus of this invention may be used for any form of cosmetic or non-cosmetic treatment of tissue, including but not limited to, hair removal, hair stimulation, treatment of skin pigmentations such as tattoo removal, treatment of port wine stains, strawberry negus, rosacea, psoriasis, the treatment of thread veins, wrinkle removal, treatment of cellulite.

In another aspect this invention provides a method for the cosmetic or non-cosmetic treatment of human or animal tissue by light, which includes the step of bringing treatment apparatus adjacent to but spaced from the tissue by a substantially unenclosed air gap a treatment device, and exposing the treatment site to light emitted by said apparatus.

Said gap is conveniently between 0.5 mm and 5 mm, preferably between 1 mm and 4 mm, and ideally between 2 mm and 3 mm.

Whilst the invention has been described above, it extends to any inventive combination of the features set out above or in the following description or claims.

The invention may be performed in various ways and, by way of example only, an embodiment thereof will now described in detail, reference being made to the accompanying drawings in which:

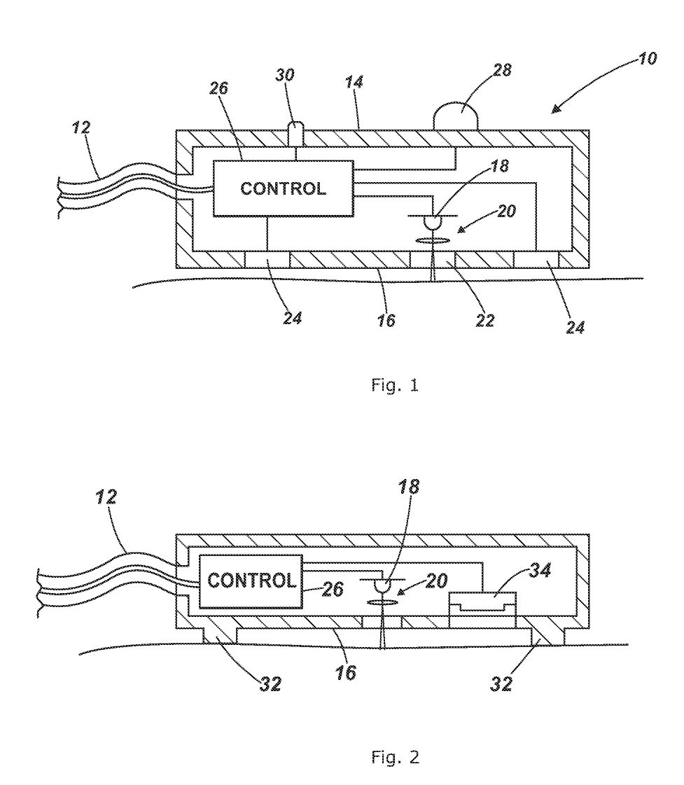

FIG. 1 is a schematic view of a first embodiment of light treatment apparatus in accordance with this invention, and

FIG. 2 is a schematic diagram of a second embodiment of light treatment apparatus in accordance with this invention.

Referring initially to FIG. 1, a light treatment wand 10 is connected to a remote console (not shown) by means of an umbilical cord 12. The wand comprises a housing 14 having a lower, skin-facing flat wall 16. The housing 14 contains a light source, for example a high intensity flash bulb 18 which directs a beam through optics indicated generally at 20 and a transparent window 22 to the surface of the skin.

The flash bulb 18 is a broadband emitter emitting light in the range of from approximately 600 nm to 1000 nm. In another embodiment the flash bulb 18 could be replaced by a laser diode emitting laser radiation or a wavelength of between 750 nm and 850 nm and preferably at about 808 nm. In both the broadband and the laser implementation the radiation is selected so that it is absorbed by melanin and so causes heating in the hair follicle.

Also mounted in the lower wall 16 are two contactless proximity sensors 24 which detect the spacing of the wall from the skin and provide this as a signal input to a control device 26 which controls operation of the light source 18. The control 26 is also connected to a firing button 28 on the upper surface of the wand and to a display LED or set of display LEDs or other visual or audible indicators 30.

The control monitors the outputs from the proximity detectors 24 and inhibits operation of the light source 18 unless an air gap is detected between the lower wall 16 and the skin. The controller may also inhibit operation of the light source if the spacing is greater than a preset amount. In addition to inhibiting operation of the light source when the air gap is less than a preset minimum or greater than a preset maximum, the control may also provide a visual or audible feedback via the display 30. In use, the wand is applied just above the surface of the skin and held there and the firing button 28 pressed; provided the air gap is within between the wall 16 and the skin is within range, the controller will allow the light source to be activated to direct a beam of light towards the skin. As the wand is moved over the skin, if the wall moves outside the predetermined range, the light source 18 is switched off until the wall comes back into range. In this manner, the wand may hover over the skin maintaining an air gap between the skin and the wall 16 thereby allowing heat transfer by radiation and convection. With practice a user may hover the wand over the skin and adjust the height to keep within the predetermined range by monitoring the display 30. If required the lower wall 16 may be provided with spacer feet which assist maintenance of the gap.

In a modified embodiment, the detectors 24 may operate both to detect the distance of the wand from the skin and also to provide a signal indicative of velocity or a change in position of the wand relative to the skin. The control circuit may be designed to be responsive to the velocity signal and to inhibit operation of the light source if the device has been stationary for longer than a preset time, or if the velocity of movement is below a preset threshold. In this manner the control circuit can operate to prevent or reduce the risk of local over-exposure and/or unwanted heat build up in the skin tissue rather than in the hair follicle. The detectors may detect velocity in the direction normal to the skin (i.e. a change in height) and/or in either or both of the other two orthogonal directions. The detectors may conveniently comprise one or more Doppler detectors directed at 45° to the axis of the light beam with one or more other Doppler detectors directed parallel to the light beam, to enable the horizontal and vertical components of any motion to be resolved.

FIG. 2 shows an arrangement where the lower wall 16 is provided with spaced feet 32, typically disposed at the four corners of a rectangular lower wall 16. In this arrangement the feet 32 hold the lower wall 16 a predetermined distance away from the skin surface, but do not significantly obstruct air flow between the lower wall 16 and the skin surface in a plane parallel to the skin. The device of FIG. 2 also includes an optical sensor 34 that may be used to provide one or more functions. The optical sensor 34 may detect the skin type and/or hair type of the skin being treated; it may detect the temperature of the skin, or it may measure distance.

1. Apparatus for the treatment of human or animal skin by light to remove hair which includes:
an applicator having a skin-facing surface;
a light source for directing light towards the skin in use;
a detector for detecting in use proximity of said skin-facing surface to the skin, and
control means responsive to said detector for causing light from said light source to be incident on the skin only when the skin-facing surface is spaced from the skin.

2. Apparatus according to claim 1, wherein said control means is responsive to enable operation of said light source only when said skin-facing surface is within a preset range of distances from the skin.

3. Apparatus according to claim 2, wherein said preset range of distances is between 0.1 mm and 10 mm.

4. Apparatus according to claim 3, wherein said preset range is between 1 mm and 4 mm.

5. Apparatus according to claim 4, wherein said preset range is between 2 mm and 3 mm.

6. Apparatus according to claim 1, including an indicator for providing an audible or visual indication of the proximity of the skin-facing surface and the skin.

7. Apparatus according to claim 1, wherein said proximity detector includes a non-contact sensor.

8. Apparatus according to claim 7, wherein said non-contact sensor is selected from the group comprising heat sensors, capacitative sensors, optical sensors, ultrasonic and sonic sensors, microwave sensors, physical sensors.

9. Apparatus according to claim 1, including a motion detector for detecting relative motion between said skin-facing surface and said skin, and said control means is responsive to said motion detector for causing light from said light source to be incident upon the skin only when relative motion between said skin-facing surface and said skin is detected.

10. Apparatus according to claim 9, wherein said control means is responsive to said motion detector to cause light from said light source to be incident upon the skin only when the magnitude of detected motion exceeds a preset threshold.

11. Apparatus for the treatment of human or animal tissue by light, which includes:
a light source, and
means for maintaining a substantially unenclosed air gap of greater than a preset distance between said apparatus and said tissue.

12. Apparatus according to claim 11, wherein said means for maintaining includes one or more spacer elements.

13. Apparatus according to claim 11, wherein said means for maintaining maintains an air gap between 0.1 mm and 10 mm.

14. Apparatus according to claim 13, wherein said means for maintaining maintains an air gap of 0.1 mm and 10 mm.

15. Apparatus according to claim 14, wherein said means for maintaining maintains an air gap of 0.1 mm and 10 mm.

16. Apparatus according to claim 1, further including means for detecting and/or classifying the nature of the tissue prior to treatment.

17. Apparatus for the treatment of human or animal tissue by light which includes:
an applicator having a tissue-facing surface;
a light source for directing light towards the tissue in use;
a detector for detecting in use proximity of said tissue-facing surface to the tissue, and
control means responsive to said detector for controlling said light source to direct light to be incident on the tissue in use only when the tissue-facing surface is spaced from the tissue.

18. A method for the cosmetic or non-cosmetic treatment of human or animal tissue by light, which includes the step of bringing treatment apparatus adjacent to but spaced from the skin by a substantially unenclosed air gap and exposing the treatment site to light emitted by said apparatus.

19. Apparatus according to claim 2, including an indicator for providing an audible or visual indication of the proximity of the skin-facing surface and the skin.

20. Apparatus according to claim 3, including an indicator for providing an audible or visual indication of the proximity of the skin-facing surface and the skin.
